(19) United States
(12) Patent Application Publication
(10) Pub. No.: US 2012/0263778 A1
(43) Pub. Date: Oct. 18, 2012

(54) POLYURETHANE UREA FOR STENT COATINGS

(75) Inventors: Jürgen Köcher, Langenfeld (DE); Christian Wamprecht, Neuss (DE); Henning Rohm, Rostock (DE); Klaus-Peter Schmitz, Rostock-Warnemunde (DE); Katrin Sternberg, Rostock (DE)

(73) Assignee: Bayer Intellectual Property GmbH, Monheim (DE)

(21) Appl. No.: 13/516,440

(22) PCT Filed: Dec. 10, 2010
(86) PCT No.: PCT/EP10/69393

§ 371 Date: Jun. 15, 2012

(51) Int. Cl. C08G 18/28; A61P 9/00; A61K 9/00; A61F 2/82; B05D 1/36; A61K 47/34

(52) U.S. Cl. 424/423; 427/407.1; 528/74; 514/772.3; 424/400

(57) ABSTRACT

The invention relates to a polyurethane urea comprising structural units of formula (I), not terminated by at least one copolymer unit of polyethylene oxide and polypropylene oxide, and that can particularly be used for producing stent coatings. The invention further relates to a substrate having a base coating made of a polyurethane urea according to the invention. The invention further relates to a coating structure comprising at least one layer comprising active substances and made of a polyurethane urea according to the invention, and at least one layer free of active substances, made of a polyurethane urea according to the invention. The invention finally relates to a method for coating a substrate, wherein at least one layer made of a polyurethane urea according to the invention is applied to the substrate.

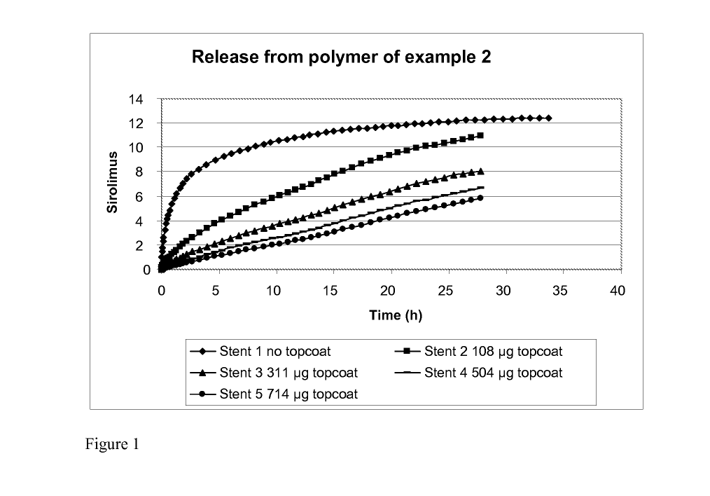

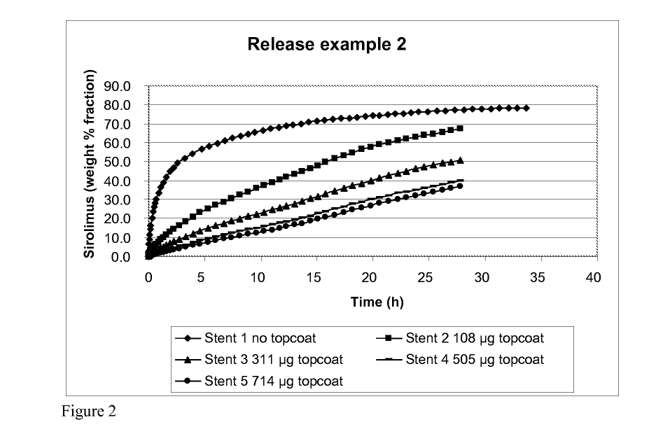

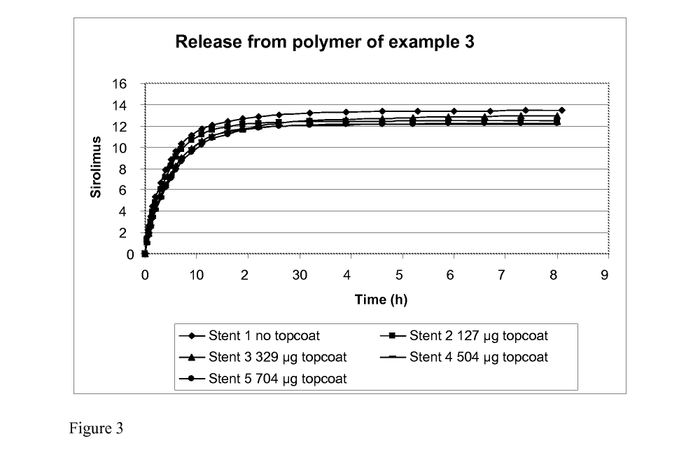

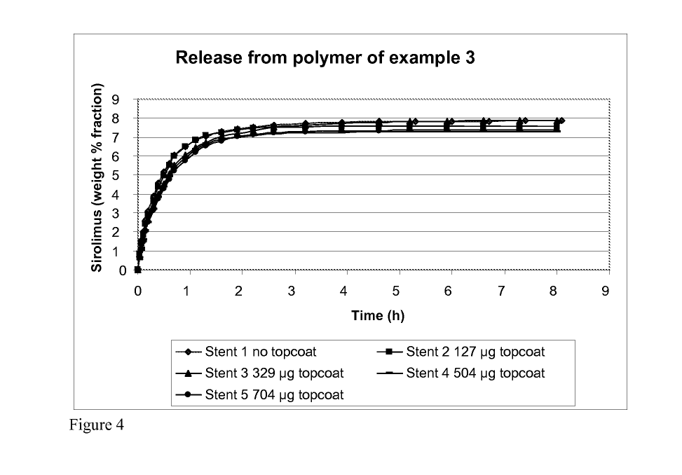

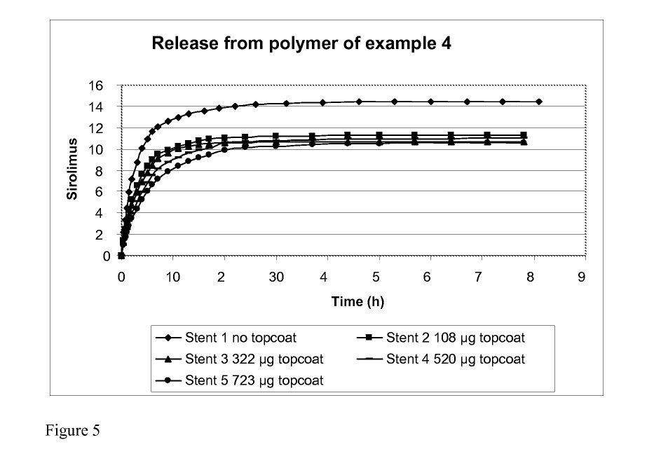

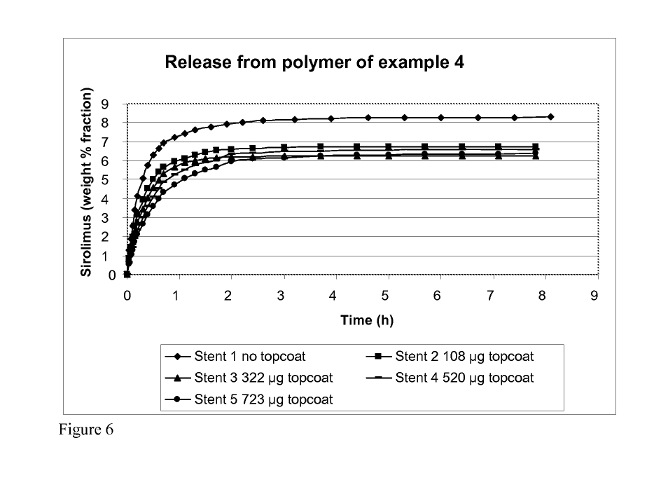

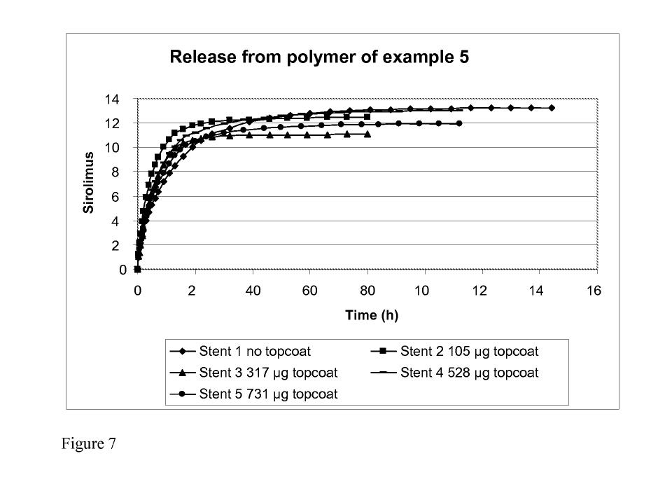

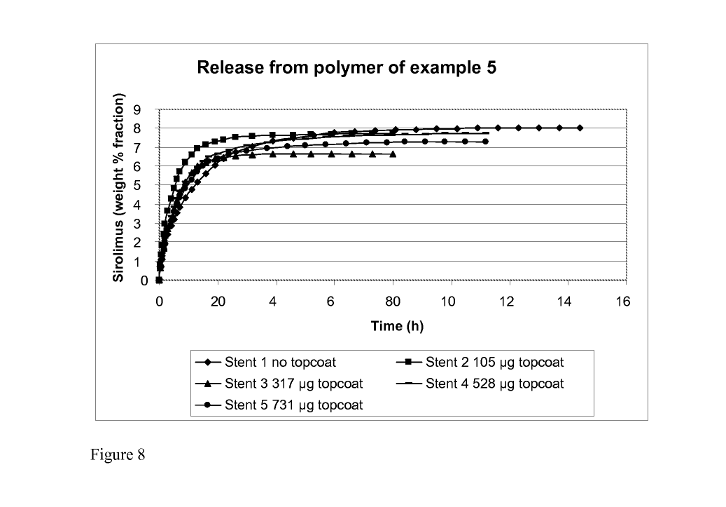

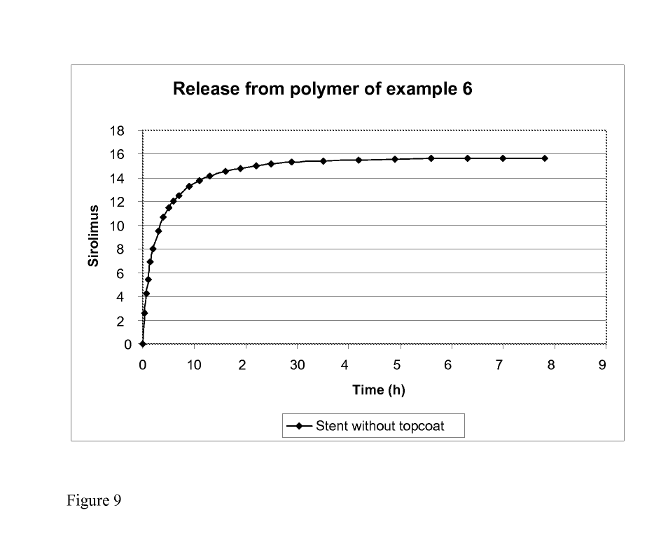

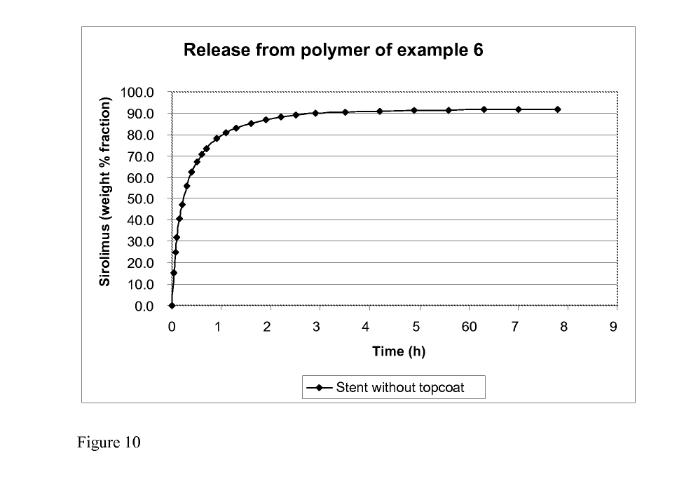

The invention relates to a polyurethane urea which can be used in particular for producing stent coatings. Additionally provided by the invention is a substrate having a basecoat comprising a polyurethane urea of the invention. Likewise provided by the invention is a layer structure comprising at least one active ingredient-containing layer comprising a polyurethane urea of the invention and at least one active ingredient-free layer comprising a polyurethane urea of the invention. Also provided by the invention lastly, is a method for coating a substrate, in which one layer of a polyurethane urea of the invention is applied to the substrate.

Polymer-based coatings for implantable articles such as stents are known in the prior art.

These coatings frequently contain active ingredients such as paclitaxel or sirolimus, the coatings being designed to release these active ingredients over a prolonged period when the stent is implanted in a body. A particular purpose of the delayed delivery of active ingredient is to reduce the risk of restenosis of the vessel undergoing treatment.

One such coated stent is described in DE 10 2005 010 998 A1, for example. Proposed therein is an active ingredient-containing coating comprising a polyurethane urea. It has emerged, however, that the delivery of the active ingredient from the polyurethane urea coating is too rapid. Hence, at the start of release (immediately after implantation) the amount of active ingredient delivered per unit time is too great, whereas at the end of the total release time the concentrations of active ingredient released are too low. Furthermore, the overall active ingredient delivery time is too short.

WO 2009/115264 A1 likewise discloses an active ingredient-containing polyurethane urea which can be used for producing coatings on stents. These polyurethane urea coatings feature good biocompatibility. Even stents provided with this coating, however, fundamentally exhibit the release kinetics already described for DE 10 2005 010 998 A1; in other words, especially at the beginning of release, the amount of active ingredient released from the coating is too great.

Active ingredient-containing polyurethane urea coatings for stents are also known from the two as yet unpublished PCT applications having the application numbers PCT/EP2009/006101 and PCT/EP2009/006102. The polyurethane ureas described therein are each terminated with a copolymer unit of polyethylene oxide and polypropylene oxide.

The polymer-based, active ingredient-containing stent coatings known in the prior art release the active ingredient they contain too rapidly and in too high an initial concentration. A consequence of this in particular is that the active ingredient is not available in the necessary concentration over the ideal target delivery period of 4 to 12 weeks.

It was an object of the invention, therefore, to provide a polyurethane urea which is suitable in particular for producing active ingredient-containing coatings for stents that following implantation release the active ingredient at a uniform delivery rate over a period of 4 to 12 weeks.

This object is achieved by means of a polyurethane urea which has structural units of the formula (I)

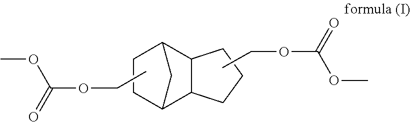

and is not terminated with at least one copolymer unit of polyethylene oxide and polypropylene oxide.

Polyurethane ureas in the sense of the present invention are polymeric compounds which have

(a) at least two repeating units containing urethane groups, of the following general structure

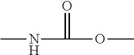

and
(b) at least one repeating unit containing urea groups

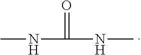

The number-average molecular weight of the polyurethane ureas is preferably 1000 to 200 000 g/mol, more preferably from 3000 to 100 000 g/mol. The number-average molecular weight here is measured against polystyrene as standard in dimethylacetamide at 30° C.

The polyurethane ureas of the invention can be prepared by reacting components which comprise at least one polycarbonate polyol component a), at least one polyisocyanate component b), at least one diamine and/or amino alcohol component c) and optionally a further polyol component d).

According to one preferred embodiment of the invention the polyurethane urea is based on a polycarbonate polyol component which preferably has an average hydroxyl functionality of 1.7 to 2.3.

The polyurethane ureas are preferably substantially linear molecules, but may also be branched, although this is less preferred. By substantially linear molecules is meant, in the context of the present invention, systems with slight incipient crosslinking, where the parent polycarbonate polyol component a) may have an average hydroxyl functionality of preferably 1.7 to 2.3, more preferably 1.8 to 2.2, very preferably 1.9 to 2.1.

The polycarbonate polyol component a) may comprise polycarbonate polyols a1) which are obtainable by reaction of carbonic acid derivatives with difunctional alcohols of the formula (II)

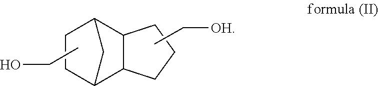

For the preparation of the polycarbonate polyol a1) it is possible, in a pressure reactor at elevated temperature, to react TCD Alcohol DM [3(4),8(9)-bis(hydroxymethyl)tricyclo[5.2.1.0/2.6]decane/tricyclodecanedimethanol] with diphenyl carbonate, dimethyl carbonate or phosgene. The reaction with dimethyl carbonate is preferred. Where dimethyl carbonate is used, the methanol elimination product is removed as a mixture with excess dimethyl carbonate by distillation.

The polycarbonate polyols a1) based on diols of the formula (II) preferably have molecular weights, as determined by the OH number, of 200 to 10 000 g/mol, more preferably of 300 to 8000 g/mol and very preferably of 400 to 6000 g/mol.

In addition it is possible for the polycarbonate polyol component a) further to the polycarbonate polyols a1) to comprise other polycarbonate polyols a2).

The polycarbonate polyols a2) may preferably comprise compounds which have an average hydroxyl functionality of 1.7 to 2.3 and a molecular weight, as determined by the OH number of 400 to 6000 g/mol and are based on hexane-1,6-diol, butane-1,4-diol or mixtures thereof.

The polycarbonate polyols a2) further preferably have molecular weights, as determined by the OH number, of 400 to 6000 g/mol, more preferably of 500 to 5000 g/mol, very preferably of 600 to 3000 g/mol. They are obtainable, for example, by reaction of carbonic acid derivatives, such as diphenyl carbonate, dimethyl carbonate or phosgene, with polyols, preferably diols. Diols contemplated in this context include, for example, ethylene glycol, 1,2- and 1,3-propanediol, 1,3- and 1,4-butanediol, 1,6-hexanediol, 1,8-octanediol, neopentylglycol, 1,4-bishydroxymethylcyclohexane, 2-methyl-1,3-propanediol, 2,2,4-trimethylpentane-1,3-diol, di-, tri- or tetraethylene glycol, dipropylene glycol, polypropylene glycols, dibutylene glycol, polybutylene glycols, bisphenol A, tetrabromobisphenol A or else lactone-modified diols.

The polycarbonate polyols a2) preferably contain 40 to 100% by weight of hexanediol, preferably 1,6-hexanediol and/or hexanediol derivatives. They preferably contain those derivatives, which as well as terminal OH groups have ether groups or ester groups. These are, for example, products obtainable by reacting 1 mol of hexanediol with at least 1 mol, preferably 1 to 2 mol of caprolactone or by etherifying hexanediol with itself to form di- or trihexylene glycol. Polyether-polycarbonate diols may be used as well. The hydroxyl polycarbonates may more particularly be substantially linear. They may also, however, be slightly branched where appropriate, as a result of the incorporation of polyfunctional components, more particularly polyols of low molecular weight. Examples of those suitable for this purpose include glycerol, hexane-1,2,6-triol, butane-1,2,4-triol, trimethylolpropane, pentaerythritol, quinitol, mannitol, sorbitol, methylglycoside or 1,3,4,6-dianhydrohexitols. Preferred polycarbonate polyols a2) are those based on hexane-1,6-diol, and also on co-diols with a modifying effect, such as butane-1,4-diol, for example or else on ε-caprolactone. Other preferred polycarbonate polyols a2) are those based on mixtures of hexane-1,6-diol and butane-1,4-diol.

In one preferred embodiment, the polycarbonate polyol component a) used is a mixture of the polycarbonate polyols a1) and those polycarbonate polyols a2) based on hexane-1,6-diol, butane-1,4-diol or mixtures thereof.

In the case of mixtures of the polycarbonate polyols a1) and a2), the fraction of a1) in the mixture is preferably at least 5 mol %, more preferably at least 10 mol %, based on the total molar amount of polycarbonate polyol.

The polyurethane ureas may additionally have units which originate from at least one polyisocyanate as synthesis component b).

As polyisocyanates b) it is possible to use all of the aromatic, araliphatic, aliphatic and cycloaliphatic isocyanates that are known to the skilled person and have an average NCO functionality≧1, preferably ≧2, individually or in any desired mixtures with one another, irrespective of whether they have been prepared by phosgene or phosgene-free processes. They may also contain iminooxadiazinedione, isocyanurate, uretdione, urethane, allophanate, biuret, urea, oxadiazinetrione, oxazolidinone, acylurea and/or carbodiimide structures. The polyisocyanates may be used individually or in any desired mixtures with one another.

It is preferred to use isocyanates from the series of the aliphatic or cycloaliphatic representatives, which have a carbon backbone (without the NCO groups present) of 3 to 30, preferably 4 to 20, carbon atoms.

Particularly preferred compounds of component b) conform to the type specified above having aliphatically and/or cycloaliphatically attached NCO groups, such as, for example, bis(isocyanatoalkyl)ethers, bis- and tris(isocyanatoalkyl)benzenes, -toluenes, and -xylenes, propane diisocyanates, butane diisocyanates, pentane diisocyanates, hexane diisocyanates (e.g., hexamethylene diisocyanate, HDI), heptane diisocyanates, octane diisocyanates, nonane diisocyanates (e.g., trimethyl-HDI (TMDI), generally as a mixture of the 2,4,4- and 2,2,4-isomers), nonane triisocyanates (e.g., 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis(isocyanatomethyl)cyclohexanes (H₆XDI), 3-iso cyanatomethyl-3,5,5-trimethylcyclohexyl isocyanate (isophorone diisocyanate, IPDI), bis(4-isocyanatocyclohexyl)methane (H₁₂MDI) or bis(isocyanatomethyl)norbornane (NBDI).

Especially preferred compounds of component b) are hexamethylene diisocyanate (HDI), trimethyl-HDI (TMDI), 2-methylpentane 1,5-diisocyanate (MPDI), isophorone diisocyanate (IPDI), 1,3- and 1,4-bis(isocyanatomethyl)cyclohexane (H₆XDI), bis(isocyanatomethyl) norbornane (NBDI), 3(4)-isocyanatomethyl-1-methylcyclohexyl isocyanate (IMCI) and/or 4,4′-bis(isocyanatocyclohexyl)methane (H₁₂MDI) or mixtures of these isocyanates. Further examples are derivatives of the above diisocyanates with uretdione, isocyanurate, urethane, allophanate, biuret, iminooxadiazinedione and/or oxadiazinetrione structure, having more than two NCO groups.

The amount of polyisocyanates b) in the preparation of the polyurethane ureas is preferably 1.0 to 3.5 mol, more preferably 1.0 to 3.3 mol and very preferably 1.0 to 3.0 mol, based in each case on the amount of compounds of the polycarbonate polyol component a).

The polyurethane ureas may contain units which originate from at least one diamine or amino alcohol as a synthesis component, and which serve as chain extenders c).

Examples of such chain extenders c) are diamines or polyamines and also hydrazides, examples being hydrazine, ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, 1,3- and 1,4-xylylenediamine, α,α,α′,α′-tetramethyl-1,3- and -1,4-xylylenediamine and 4,4′-diaminodicyclohexylmethane, dimethylethylenediamine, adipic dihydrazide, 1,4-bis(aminomethyl)cyclohexane, 4,4′-diamino-3,3′-dimethyldicyclohexylmethane and other (C₁-C₄) di- and tetraalkyldicyclohexylmethanes, e.g., 4,4′-diamino-3,5-diethyl-3′,5′-diisopropyldicyclohexylmethane.

Generally contemplated as diamines or amino alcohols are low molecular weight diamines or amino alcohols which contain active hydrogen of differing reactivity toward NCO groups, such as compounds which as well as a primary amino group also contain secondary amino groups, or as well as an amino group (primary or secondary) also contain OH groups. Examples of such compounds are primary and secondary amines, such as 3-amino-1-methylaminopropane, 3-amino-1-ethylaminopropane, 3-amino-1-cyclohexylaminopropane, 3-amino-1-methylaminobutane, and also amino alcohols, such as N-aminoethylethanolamine, ethanolamine, 3-aminopropanol, neopentanolamine and, more preferably, diethanolamine.

Constituent c) of the polyurethane ureas may be used as a chain extender during their preparation.

The amount of constituent c) in preparing the polyurethane ureas is preferably 0.1 to 1.5 mol, more preferably 0.2 to 1.3 mol, more particularly 0.3 to 1.2 mol, based in each case on the amount of the compounds of component a).

In a further embodiment the polyurethane ureas comprise additional units which originate from at least one further polyol d) as a synthesis component.

The other low molecular mass polyols d) used for synthesizing the polyurethane ureas generally have the effect of stiffening and/or branching the polymer chain. The molecular weight is preferably 62 to 500 g/mol, more preferably 62 to 400 g/mol, more particularly 62 to 200 g/mol.

Suitable polyols may contain aliphatic, alicyclic or aromatic groups. Examples that may be mentioned here include the low molecular weight polyols having up to about 20 carbon atoms per molecule, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentylglycol, hydroquinone dihydroxyethyl ether, bisphenol A (2,2-bis(4-hydroxyphenyl)propane), hydrogenated bisphenol A (2,2-bis(4-hydroxycyclohexyl)propane), and also trimethylolpropane, glycerol or pentaerythritol and mixtures of these and optionally also other low molecular weight polyols. Esterdiols may be used as well, such as, for example α-hydroxybutyl-ε-hydroxycaproic ester, ω-hydroxyhexyl-γ-hydroxybutyric ester, (β-hydroxyethyl) adipate or bis(β-hydroxyethyl)terephthalate.

The amount of constituent d) in preparing the polyurethane ureas is preferably 0.05 to 1.0 mol, more preferably 0.05 to 0.5 mol, more particularly 0.1 to 0.5 mol, based in each case on the amount of the compounds of the polycarbonate polyol component a).

The reaction of the isocyanate-containing component b) with the hydroxy- or amine-functional compounds a), c) and optionally d) takes place typically subject to a slight NCO excess over the reactive hydroxy or amine compounds. At the endpoint of the reaction, through attainment of a target viscosity, there are always residues of active isocyanates still remaining. These residues must be blocked in order that no reaction takes place with large polymer chains. Any such reaction leads to three-dimensional crosslinking and to the gelling of the batch. A solution of that kind can no longer be processed. The batches typically contain large amounts of alcohols. Within a number of hours of standing or stirring of the batch at room temperature, these alcohols block the remaining isocyanate groups.

Where the residual isocyanate content was blocked during the preparation of the polyurethane ureas, these ureas also have, as synthesis components e), monomers which are located at each of the chain ends, capping them.

These synthesis components e) derive on the one hand from monofunctional compounds that are reactive with NCO groups, such as monoamines, more particularly from mono-secondary amines or monoalcohols. Mention may be made, here, of ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof.

Since synthesis components e) are used substantially in the polyurethane ureas for destroying the NCO excess, the amount required is dependent essentially on the amount of the NCO excess, and cannot be specified generally.

It is preferred to forego synthesis component e) during the synthesis. In that case, unreacted isocyanate is reacted preferably to form terminal urethanes by the solvent alcohols that are present in very large concentrations.

For the preparation of the polyurethane solutions of the invention, the polycarbonate polyol component a), the polyisocyanate b) and optionally the polyol d), are reacted in the melt or in solution until all of the hydroxyl groups have been consumed.

The stoichiometry used in this case between the individual components participating in the reaction is a consequence of the aforementioned quantitative proportions.

The reaction takes place at a temperature of preferably 60 to 110° C., more preferably 75 to 110° C., more particularly 90 to 110° C., with temperatures of 110° C. being preferred on account of the reaction rate. Higher temperatures may likewise be employed, but then, in certain cases, and depending on the individual components used, there is a risk of decomposition processes and instances of discoloration occurring in the resulting polymer.

For the prepolymer formed from isocyanate and all of the components containing hydroxyl groups, reaction in the melt is preferred, although the risk exists that the fully reacted mixtures will have high viscosities. In such cases it is also advisable to add solvents. However, as far as possible not more than approximately 50% by weight of solvent should be present, since otherwise the dilution significantly retards the reaction rate.

In the case of the reaction of components containing isocyanate and hydroxyl groups, the reaction may take place in the melt within a period of 1 hour to 24 hours. Small additions of solvent quantities result in a deceleration, but the reaction periods lie within the same periods.

The sequence of the addition/reaction of the individual components may deviate from the sequence indicated above. This may be especially advantageous when the mechanical properties of the coatings producible from the polyurethane urea are to be altered. If, for example, all of the components containing hydroxyl groups are reacted simultaneously, a mixture of hard and soft segments is formed. If, for example, the low molecular weight polyol is added after the polycarbonate polyol component, defined blocks are obtained, and this may result in different properties on the part of the resultant coatings. The present invention is therefore not limited to any particular sequence of the addition/reaction of the individual components.

After these reaction steps, further solvent can be added and optionally dissolved chain extender diamine or dissolved chain extender amino alcohol (component (c)) can be added.

The further addition of the solvent takes place preferably in steps, in order not unnecessarily to slow down the reaction, as would occur, for example, at the beginning of the reaction if the amount of solvent were to be added completely. Furthermore, a high solvent content of the beginning of the reaction imposes a relatively low temperature, which is at least co-determined by the nature of the solvent. This too leads to a deceleration of the reaction.

After the target viscosity has been reached, the remaining residues of NCO can be blocked by a monofunctional aliphatic amine. The isocyanate groups still remaining are preferably blocked by reaction with the alcohols present in the solvent mixture.

The polyurethane ureas of the invention may further comprise additives and constituents that are customary for the particular desired end use.

One example of such are active pharmacological ingredients and additives which promote the release of active pharmacological ingredients (“drug-eluting additives”). In one preferred embodiment, the polyurethane urea comprises active pharmacological ingredients.

Active pharmacological ingredients which may be used in coatings on medical devices are, for example, thromboresistant agents, antibiotic agents, antitumor agents, growth hormones, antiviral agents, antiangiogenic agents, angiogenic agents, antimitotic agents, anti-inflammatory agents, cell cycle regulators, genetic agents, hormones, and also their homologs, derivatives, fragments, pharmaceutical salts and combinations thereof.

Specific examples of active pharmacological ingredients hence include thromboresistant (non thrombogenic) agents and other agents for suppressing acute thrombosis, stenosis or late restenosis of the arteries. Examples of these are heparin, streptokinase, urokinase, tissue plasminogen activator, anti-thromboxan-B₂ agent; anti-B thromoboglobulin, prostaglandin-E, aspirin, dipyridimol, anti-thromboxan-A₂ agent, murine monoclonal antibody 7E3, triazolopyrimidine, ciprostene, hirudin, ticlopidine, nicorandil etc.

A growth factor may likewise be used as an active pharmacological ingredient in order to suppress subintimal fibromuscular hyperplasia at the arterial stenosis site, or any other cell growth inhibitor may be used at the stenosis site.

The active pharmacological ingredient may also consist of a vasodilator, in order to counteract vasospasm. This may be, for example, an anti-spasm agent such as papaverine.

The active pharmacological ingredient may be a vasoactive agent per se such as calcium antagonists or α- and β-adrenergic agonists or antagonists. Additionally the active pharmacological ingredient may be a biological adhesive such as medical-grade cynoacrylate, or fibrin.

The active pharmacological ingredient may additionally be an antineoplastic agent such as 5-fluorouracil, preferably with a controlling releasing vehicle for the agent, as for example for the use of an ongoing controlled releasing antineoplastic agent at a tumor site.

The active pharmacological ingredient may be an antibiotic, preferably in combination with a controlling releasing vehicle for ongoing release from the coating of a medical device at a localized focus of infection within the body. Similarly, the active pharmacological ingredient may comprise steroids for the purpose of suppressing inflammation in localized tissue, or for other reasons.

Specific examples of suitable active pharmacological ingredients include the following:

(a) heparin, heparin sulfate, hirudin, hyaluronic acid, chondroitin sulfate, dermatan sulfate, keratan sulfate, lytic agents, including urokinase and streptokinase, their homologs, analogs, fragments, derivatives and pharmaceutical salts thereof;
(b) antibiotic agents such as penicillins, cephalosporins, vacomycins, aminoglycosides, quinolones, polymyxines, erythromycins; tetracyclines, chloramphenicols, clindamycins, lincomycins, sulfonamides, their homologs, analogs, derivatives, pharmaceutical salts and mixtures thereof'
(c) paclitaxel, docetaxel, immunosuppressants such as sirolimus or sirolimus-related limus derivatives such as for example, everolimus, biolimus A9, tacrolimus or zotarolimus, alkylating agents including, mechlorethamine, chlorambucil, cyclophosphamide, melphalane and ifosfamide; antimetabolites, including, methotrexate, 6-mercaptopurine, 5-fluorouracil and cytarabine; plant alkaloids, including vinblastine; vincristine and etoposide; antibiotics including, doxorubicin, daunomycin, bleomycin and mitomycin; nitrosurea, including carmustine and lomustine; inorganic ions including cisplatin; biological reaction modifiers, including interferon; angiostatins and endostatins; enzymes, including asparaginase; and hormones, including tamoxifen and flutamide, their homologs, analogs, fragments, derivatives, pharmaceutical salts and mixtures thereof;
(d) antiviral agents such as amantadine, rimantadine, rabavirin, idoxuridine, vidarabin, trifluridine, acyclovir, ganciclovir, zidovudine, phosphonoformates, interferons, their homologs, analogs, fragments, derivatives, pharmaceutical salts and mixtures thereof; and
(e) antiinflammatories such as, for example ibuprofen, dexamethasone or methylprednisolone.

The invention further provides a substrate having applied thereon a basecoat comprising a polyurethane urea of the invention.

Applied on the basecoat there may preferably be a topcoat comprising a polyurethane urea of the invention, which differs in its chemical and/or physical properties from the basecoat.

The basecoat may, more particularly comprise an active pharmacological ingredient.

The topcoat may contain a significantly lower concentration of active ingredient than the basecoat, i.e. for example, less than 10% of the amount of active ingredient present per unit volume in the basecoat. It is particularly preferred if the topcoat is active ingredient-free or virtually active ingredient-free. The presence of the topcoat further decelerates the delivery of the active ingredient from the basecoat.

In one particularly preferred embodiment of the substrate of the invention, the basecoat has a thickness of 5 to 20 μm and/or the topcoat has a thickness of 0.5 to 10 μm.

The substrate may more particularly be a medical device.

The term “medical device” is to be understood broadly in the context of the present invention. Suitable, nonlimiting examples of medical devices are contact lenses; cannulas; catheters, as for example urological catheters such as urinary catheters or urethral catheters; central venous catheters; venous catheters or inlet or outlet catheters; dilation balloons; catheters for angioplasty and biopsy; catheters used for introducing a stent, an embolism filter or a vena cava filter; balloon catheters or other expandable medical devices; endoscopes; laryngoscopes; tracheal devices such as endotracheal tubes; respirators and other tracheal aspiration devices; bronchoalveolar lavage catheters; catheters used in coronary angioplasty; guide rods, insertion guides and the like; vascular plugs; pacemaker components; cochlear implants; dental implant tubes for feeding, drainage tubes; and guide wires.

The polyurethane ureas of the invention may be used, furthermore for producing coatings, as for example for gloves, stents and other implants; extracorporeal blood lines; membranes, as for example for dialysis; blood filters; devices for circulatory support; dressing material for wound management; urine bags and stoma bags. Also included are implants which comprise a medically active agent, such as medically active agents for stents or for balloon surfaces or for contraceptives.

With very particular preference the medical device is an implantable device and more particularly a stent.

Yet a further subject provided by the invention is a layer structure comprising at least one active ingredient-containing layer comprising a polyurethane urea of the invention, and at least one active ingredient-free layer comprising a polyurethane urea of the invention.

A method for coating a substrate, in which at least one layer of a polyurethane urea of the invention is applied to the substrate, is likewise provided by the invention.

In this method, preferably a basecoat comprising an active-ingredient containing polyurethane urea is applied to the substrate, and a topcoat comprising an active ingredient-free polyurethane urea is applied to the basecoat.

The invention also provides a substrate produced by the method of the invention.

### EXAMPLES

The invention is elucidated in detail below by means of examples.

### Methods:

The NCO content of the resins described in the inventive and comparative examples was determined by titration in accordance with DIN EN ISO 11909.

The solids contents were determined according to DIN-EN ISO 3251. For this purpose 1-1.5 g of polyurethane urea solution were dried to constant weight in a vacuum drying cabinet at 50° C. for around 17 hours.

Unless noted otherwise, the quantity figures indicated in % are understood to be % by weight and are based on the organic polyurethane urea solution obtained.

The OH numbers were determined according to DIN 53240.

Viscosity measurements were carried out using the Physics MCR 51 rheometer from Anton Paar GmbH, Ostfildern, Germany.

### Substances Used and Abbreviations:

Desmophen C2200: polycarbonate polyol, OH number 56 mg KOH/g, number-average molecular weight 2000 g/mol (Bayer MaterialScience AG, Leverkusen, Del.)
Polyether LB 25: monofunctional polyether based on ethylene oxide/propylene oxide, with number-average molecular weight of 2250 g/mol, OH number 25 mg KOH/g (Bayer MaterialScience AG, Leverkusen, Del.)
TCD Alcohol DM 3 (4),8(9)-bis(hydroxymethyl)tricyclo-[5.2.1.0/2.6]decane/tricyclodecanedimethanol, Celanese Corp., Dallas, USA
Sirolimus: sirolimus (from Streptomyces hygroscopicus; CAS: 53123-88-9; Poli Industria Chimica S.p.A; Rozzano, Italy)
Stents used: CoCr stents for coronary indication, from the ProKinetik Stents Series from Biotronik (Berlin, Germany) without the standard Si carbide coating, 18 mm long with 60 μm wall thickness and a total surface area of 76.56 mm².

### Example 1

Preparation of a cycloaliphatic polycarbonate diol based on TCD Alcohol DM with a number-average molecular weight of 1300 g/mol

A 16 l pressure reactor with top-mounted distillation attachment, stirrer, and receiver was charged with 5436 g of TCD Alcohol DM including 1.2 g of yttrium(III) acetylacetonate and also with 3810 g of dimethyl carbonate at 80° C. The reaction mixture was then heated to 135° C. under a nitrogen atmosphere over 2 hours and was held at that temperature with stirring for 24 hours, during which the pressure rose to 6.3 bar (absolute). It was then cooled to 60° C., and air was admitted. The methanol elimination product was subsequently removed by distillation in a mixture with dimethyl carbonate, the temperature being raised in steps to 150° C. Stirring was continued at 150° C. then for 4 hours more, followed by heating to 180° C. and then by stirring at 180° C. for 4 hours again. The temperature was subsequently reduced to 90° C. and a stream of nitrogen (5 l/h) was passed through the reaction mixture, while the pressure was lowered to 20 mbar. Thereafter the temperature was raised to 180° C. over 4 hours and held there for 6 hours. During this time, further methanol in a mixture with dimethyl carbonate was removed from the reaction mixture.

Following admission of air and cooling of the reaction batch to room temperature, a yellowish, solid polycarbonate diol was obtained which had the following characteristics:

Mₙ=1290 g/mol; OH number=87 mg KOH/g;

### Example 2

### Inventive

An amount of 97.8 g of Desmophen C 2200, 63.6 g of polycarbonate diol from example 1, and 47.8 g of 4,4′-bis(isocyanatocyclohexyl)methane (H₁₂MDI) were reacted at 110° C. for 4 hours until the NCO content was constant at 3.3%. The mixture was allowed to cool and was diluted with 335 g of toluene and 185 g of isopropanol. At room temperature, a solution of 12.6 g of isophoronediamine in 92.0 g of 1-methoxypropan-2-ol was added. After the end of the increase in molecular weight and attainment of the desired viscosity range, stirring was continued at room temperature for 15 hours, in order to block the residual isocyanate content with isopropanol. This gave 833.8 g of a 27.0% strength polyurethane urea solution in toluene/isopropanol/1-methoxypropan-2-ol having a viscosity of 46 400 mPas at 23° C.

### Example 3

### Comparative

This example describes the synthesis of a hydrophobic coating without addition of the polycarbonate diol of example 1.

An amount of 195.4 g of Desmophen C 2200, and 47.8 g of 4,4′-bis(isocyanatocyclohexyl)methane (H₁₂MDI) were reacted at 110° C. for 17 hours until the NCO content was constant at 2.8%. The mixture was allowed to cool and was diluted with 350 g of toluene and 200 g of isopropanol. At room temperature, a solution of 11.5 g of isophoronediamine in 85.0 g of 1-methoxypropan-2-ol was added. After the end of the increase in molecular weight and attainment of the desired viscosity range, stirring was continued at room temperature for 20 hours, in order to block the residual isocyanate content with isopropanol. This gave 889.7 g of a 29.3% strength polyurethane urea solution in toluene/isopropanol/1-methoxypropan-2-ol having a viscosity of 24 600 mPas at 23° C.

### Example 4

### Comparative

This example describes the synthesis of a hydrophilic coating without addition of the polycarbonate diol of example 1.

An amount of 195.4 g of Desmophen C 2200, 40.0 g of LB 25, and 47.8 g of 4,4′-bis(isocyanatocyclohexyl)methane (H₁₂MDI) were reacted at 110° C. for 19 hours until the NCO content was constant at 2.2%. The mixture was allowed to cool and was diluted with 350 g of toluene and 200 g of isopropanol. At room temperature, a solution of 12.0 g of isophoronediamine in 100 g of 1-methoxypropan-2-ol was added. After the end of the increase in molecular weight and attainment of the desired viscosity range, stirring was continued for 4 hours, in order to block the residual isocyanate content with isopropanol. This gave 945.2 g of a 31.6% strength polyurethane urea solution in toluene/isopropanol/1-methoxypropan-2-ol having a viscosity of 19 300 mPas at 23° C.

### Example 5

### Comparative

This example describes the synthesis of a hydrophilic coating with addition of the polycarbonate diol of example 1.

An amount of 97.8 g of Desmophen C 2200, 63.6 g of polycarbonate diol from example 1, 30.0 g of LB 25 and 47.8 g of 4,4′-bis(isocyanatocyclohexyl)methane (H₁₂MDI) were reacted at 110° C. for 2 hours until the NCO content was constant at 2.6%. The mixture was allowed to cool and was diluted with 335 g of toluene and 185 g of isopropanol. At room temperature, a solution of 12.0 g of isophoronediamine in 94.0 g of 1-methoxypropan-2-ol was added. After the end of the increase in molecular weight and attainment of the desired viscosity range, stirring was continued for 15 hours, in order to block the residual isocyanate content with isopropanol. This gave 865.2 g of a 31.0% strength polyurethane urea solution in toluene/isopropanol/1-methoxypropan-2-ol having a viscosity of 33 300 mPas at 23° C.

### Example 6

### Comparative

An amount of 586.2 g of Desmophen C 2200, 21.6 g of butane-1,4-diol and 141.3 g of 4,4′-bis(isocyanatocyclohexyl)methane (H₁₂MDI) are reacted at 150° C. for 140 minutes. The hot reaction mixture is poured out into a dish preheated to 80° C., and is stored in a drying cabinet at 90° C. for 2 hours. Cooling produces a solid product, which in order to coat stents must be taken up in solvent. For this purpose, 30 g of the solid product obtained are introduced into 70 g of toluene/isopropanol mixture (64% by weight toluene, 36% by weight isopropanol) and stirred at room temperature for 4 hours. This gives a clear solution, without undissolved constituents, having a viscosity of 640 mPas at 23° C.

### Example 7

### Production of the Coating Solution for Stent Coating

The polyurethane stock solution from example 2, with a polymer fraction of 27.0% by weight, was diluted in a ratio of 1:80 with a mixture of 54% toluene and 46% 2-propanol, to give a polymer fraction of ˜0.34% by weight. The diluted solution was admixed with 15% by weight of the active ingredient (sirolimus or paclitaxel) based on the polymer mass, as a methanolic solution (˜5 mg/ml). For this purpose, for a coating solution, 0.5 g of the polyurethane stock solution was weighed out into an Erlenmeyer flask, and 40 g of the toluene/2-propanol mixture was added for dilution with stirring. Then 20 mg of paclitaxel or sirolimus were dissolved in 4 ml of methanol, and added, likewise with stirring.

In the same way, the polyurethane stock solutions from examples 3, 4, 5 and 6 were diluted and likewise admixed with 15% by weight of the active ingredient, based on the polymer mass.

### Example 8

### General Method for Stent Coating

### a) Single-Coat System

Prior to coating, the stents were cleaned with chloroform in an ultrasound bath. The cleaned stents were then inspected under a light microscope, and cleaned again where necessary. The initial mass of the uncoated stents was determined using an ultra-micro-balance.

The stents were coated by means of a spray coating unit. The basis of this coating technique is that a coating solution as per example 7 is atomized by compressed air in a nozzle with a spraying pressure of 0.3-0.5 bar. The internal diameter of the spraying nozzle used may be between 0.1 and 3 mm. The stent to be coated here is located in a mount which is positioned in the spray jet and which rotates the stent about its longitudinal axis. The distance between stent and nozzle may be between 10 and 100 mm. The progress of the coating procedure here is determined by weighing the stents and calculating the difference relative to the initial masses. After complete coating has taken place, the stents are dried in a vacuum drying cabinet at 40° C. under a pressure of approximately 10 mbar for between 12 hours and 24 hours.

### b) Multiple-Coat System

A first basecoat consists of the dilute polymer stock solutions described in example 7 (prepared from the polyurethane solutions of examples 2-6), to which the amounts of sirolimus specified in example 7 were added. As described in the preceding paragraph, stents were coated with these active-ingredient containing polyurethane solutions, and dried as indicated. Then, in a second operation, the pure diluted polyurethane solutions from example 7 without a sirolimus fraction were applied as a topcoat to the dried, active ingredient-containing polyurethane coating, and likewise dried as indicated. The topcoat taken in each case was the same polyurethane solution also used as the active ingredient-containing matrix.

### c) Coat Thickness Determination

Using a confocal laser microscope (Olympus LEXT OLS 300), measurements for determining the coat thickness of the applied polymer/active ingredient coats were carried out on stents coated in the manner described. With the selected basecoat masses of 1000-1100 μg, coat thicknesses of 6 μm to 14 μm are found at various measurement points on a stent. A topcoat with a coating mass of 100 μg produces a coat thickness of 1.2 μm to 1.4 μm. Topcoats with a coating mass of 700 μg produce coat thicknesses of 8 μm to 9 μm.

### Example 9

### General Method for Investigating Drug Release

The stents coated as per example 8 were crimped manually onto a balloon catheter (balloon catheter from the stent system Lekton 3.0/20, from Biotronik, Berlin, Germany). The crimped stent was immersed in each case into a glass vial which can be closed with a screw lid and in which 2 ml of a 0.9% strength NaCl_{[aq]} solution heated to 37° C. (additionally containing 0.05% by weight of nonionic detergent Brij 35 and 3 mg/l of antioxidant BHT (butylated hydroxytoluene) had been introduced, and was then dilated with the aid of a manual pump (Guidant, Boston Scientific) at a pressure of 10 bar. The glass vial was sealed and shaken slowly with a shaker (IKA MS 3 basic) at 37° C. After a time defined beforehand, the stent was removed from the elution medium and dried on a tissue. The stent was then replaced in a vial with 2 ml of elution medium and shaken at 37° C.

The amount of active ingredient released was determined by means of an HPLC apparatus (Knauer Berlin; UV detector K-2501; HPLC pump K-1001; solvent organizer K-1500; Smartline Autosampler 3800; Jet Stream oven, Eurospher-100 column, C18, 120×4 mm) At a flow rate of 1 ml/min, a mixture of ultrapure water and acetonitrile (35/65; v/v) was used as mobile phase for sirolimus, whereas for paclitaxel a mixture of acetonitrile and an aqueous KH₂PO₄ solution (pH=3.5) (50/50, v/v) was used. During measurement, the UV detector was set at a measuring wavelength of 254 nm.

### Example 10

### Results of the Release Studies

The aim of the invention is to develop a stent coating which releases the active ingredient sirolimus continuously over a number of weeks. For this purpose, in accordance with the instructions in examples 7-9, stents were produced which as well as active ingredient-containing polyurethane basecoat also contain increasing amounts of active ingredient-free polyurethane topcoat. The tables set out below contain the release rates of sirolimus from active ingredient-containing polyurethane coatings without active ingredient-free topcoats and also with increasing masses of active ingredient-free topcoats.

For each coating, two tables and graphs are shown: the release of the absolute amount of sirolimus, and also the release as a percentage fraction of the sirolimus employed. The values constitute the quantities released cumulatively at the time in question.

1. Stents with Coating of Polyurethane from Example 2 (Inventive)

| | Basecoat (μg) | Sirolimus (μg) | Topcoat (μg) |
|---|---|---|---|
| Stent 1 | 1058 | 158.7 | 0 |
| Stent 2 | 1081 | 162.15 | 108 |
| Stent 3 | 1063 | 159.45 | 311 |
| Stent 4 | 1113 | 166.95 | 504 |
| Stent 5 | 1048 | 157.2 | 714 |

| | Stent 1 | | Stent 2 | | Stent 3 | | Stent 4 | | Stent 5 |
|---|---|---|---|---|---|---|---|---|---|
| | Total | | Total | | Total | | Total | | Total |
| Time | abs. | Time | abs. | Time | abs. | Time | abs. | Time | abs. |
| (h) | (μg) | (h) | (μg) | (h) | (μg) | (h) | (μg) | (h) | (μg) |
| 0 | 0 | 0 | 0 | 0 | 0 | 0 | 0 | 0 | 0 |
| 0.33 | 9.836 | 0.33 | 1.836 | 0.33 | 0.704 | 0.33 | 0.204 | 0.33 | 0.152 |
| 0.67 | 14.324 | 0.67 | 2.508 | 0.67 | 1.024 | 0.67 | 0.32 | 0.67 | 0.268 |
| 1 | 17.924 | 1 | 3.036 | 1 | 1.208 | 1 | 0.476 | 1 | 0.372 |
| 1.5 | 22.64 | 1.5 | 3.744 | 1.5 | 1.572 | 1.5 | 0.648 | 1.5 | 0.52 |
| 2 | 26.016 | 2 | 4.644 | 2 | 1.992 | 2 | 0.86 | 2 | 0.664 |
| 3 | 32.132 | 3 | 5.948 | 3 | 2.632 | 3 | 1.184 | 3 | 0.876 |
| 4 | 37.132 | 4 | 7.12 | 4 | 3.248 | 4 | 1.512 | 4 | 1.14 |
| 5 | 41.164 | 5 | 8.252 | 5 | 3.832 | 5 | 1.84 | 5 | 1.388 |
| 6 | 44.652 | 6 | 9.312 | 6 | 4.396 | 6 | 2.196 | 6 | 1.608 |
| 7 | 47.864 | 7 | 10.324 | 7 | 4.98 | 7 | 2.496 | 7 | 1.848 |
| 9 | 53.184 | 9 | 12.18 | 9 | 6.056 | 9 | 3.128 | 9 | 2.32 |
| 11 | 57.876 | 11 | 14.024 | 11 | 7.148 | 11 | 3.756 | 11 | 2.82 |
| 13 | 61.98 | 13 | 15.68 | 13 | 8.104 | 13 | 4.384 | 13 | 3.256 |
| 16 | 66.528 | 16 | 18.204 | 16 | 9.52 | 16 | 5.368 | 16 | 3.904 |
| 19 | 70.744 | 19 | 20.488 | 19 | 10.852 | 19 | 6.32 | 19 | 4.612 |
| 22 | 74.212 | 22 | 22.816 | 22 | 12.304 | 22 | 7.28 | 22 | 5.364 |
| 26 | 78.256 | 27 | 26.288 | 27 | 14.184 | 27 | 8.628 | 27 | 6.376 |
| 32 | 82.144 | 33 | 29.648 | 33 | 16.164 | 33 | 10.124 | 33 | 7.584 |
| 39 | 85.716 | 40 | 33.696 | 40 | 18.624 | 40 | 12.036 | 40 | 8.972 |
| 47 | 89.468 | 46 | 37.124 | 46 | 20.872 | 46 | 13.784 | 46 | 10.336 |
| 54 | 92.248 | 53 | 40.308 | 53 | 23.196 | 53 | 15.492 | 53 | 11.776 |
| 61 | 94.728 | 60 | 43.78 | 60 | 25.516 | 60 | 17.404 | 60 | 13.152 |
| 68 | 97.004 | 67 | 46.62 | 67 | 27.424 | 67 | 18.976 | 67 | 14.356 |
| 75 | 98.972 | 74 | 49.52 | 74 | 29.484 | 74 | 20.36 | 74 | 15.764 |
| 82 | 100.88 | 81 | 52.5 | 81 | 31.56 | 81 | 21.892 | 81 | 17.024 |
| 89 | 102.46 | 89 | 55.416 | 89 | 33.656 | 89 | 23.628 | 89 | 18.368 |
| 95 | 103.8 | 96 | 58.032 | 96 | 35.488 | 96 | 25.02 | 96 | 19.624 |
| 102 | 105.252 | 103 | 60.316 | 103 | 37.232 | 103 | 26.372 | 103 | 20.764 |
| 109 | 106.636 | 110 | 62.72 | 110 | 38.904 | 110 | 27.908 | 110 | 21.992 |
| 116 | 107.724 | 117 | 65.24 | 117 | 40.712 | 117 | 29.292 | 117 | 23.416 |
| 123 | 109.012 | 123 | 67.476 | 123 | 42.456 | 123 | 30.776 | 123 | 24.636 |
| 129 | 110.108 | 130 | 69.84 | 130 | 44.172 | 130 | 32.236 | 130 | 25.92 |
| 136 | 111.12 | 137 | 72.416 | 137 | 46.076 | 137 | 33.876 | 137 | 27.392 |
| 143 | 112.132 | 144 | 75.2 | 144 | 48.24 | 144 | 35.66 | 144 | 29.136 |
| 150 | 112.916 | 151 | 77.74 | 151 | 50.496 | 151 | 37.536 | 151 | 30.708 |
| 157 | 113.716 | 158 | 80.572 | 158 | 52.76 | 158 | 39.524 | 158 | 32.552 |
| 164 | 114.388 | 166 | 83.588 | 166 | 54.908 | 166 | 41.716 | 166 | 34.392 |
| 172 | 115.144 | 174 | 86.092 | 174 | 57.064 | 174 | 43.652 | 174 | 36.192 |
| 179 | 115.836 | 182 | 88.844 | 182 | 59.344 | 182 | 45.652 | 182 | 38.284 |
| 186 | 116.424 | 190 | 91.396 | 190 | 61.548 | 190 | 47.844 | 190 | 40.276 |
| 193 | 116.996 | 198 | 93.296 | 198 | 63.556 | 198 | 49.748 | 198 | 41.972 |
| 200 | 117.58 | 206 | 95.316 | 206 | 65.888 | 206 | 51.756 | 206 | 43.804 |
| 206 | 118.076 | 214 | 97.332 | 214 | 68.016 | 214 | 53.556 | 214 | 45.716 |
| 213 | 118.544 | 222 | 99.208 | 222 | 70.008 | 222 | 55.508 | 222 | 47.508 |
| 220 | 119.084 | 230 | 100.72 | 230 | 71.656 | 230 | 56.964 | 230 | 49.032 |
| 227 | 119.608 | 238 | 102.112 | 238 | 73.456 | 238 | 58.608 | 238 | 50.604 |
| 234 | 120.112 | 246 | 103.348 | 246 | 75.056 | 246 | 60.064 | 246 | 51.94 |
| 241 | 120.616 | 254 | 104.768 | 254 | 77.064 | 254 | 61.8 | 254 | 53.472 |
| 249 | 121.18 | 262 | 106.276 | 262 | 78.3 | 262 | 63.384 | 262 | 54.844 |
| 257 | 121.608 | 270 | 107.92 | 270 | 79.504 | 270 | 65.028 | 270 | 56.256 |
| 265 | 122.044 | 278 | 109.516 | 278 | 80.676 | 278 | 66.764 | 278 | 57.84 |
| 273 | 122.384 | | | | | | | | |
| 281 | 122.676 | | | | | | | | |
| 289 | 122.988 | | | | | | | | |
| 297 | 123.316 | | | | | | | | |
| 305 | 123.536 | | | | | | | | |
| 313 | 123.764 | | | | | | | | |
| 321 | 123.92 | | | | | | | | |
| 329 | 124.12 | | | | | | | | |
| 337 | 124.248 | | | | | | | | |
| | Stent 1 | | Stent 2 | | Stent 3 | | Stent 4 | | Stent 5 |
| Time | fraction | Time | fraction | Time | fraction | Time | fraction | Time | fraction |
| (h) | (wt. %) | (h) | (wt. %) | (h) | (wt. %) | (h) | (wt. %) | (h) | (wt. %) |
| 0 | 0.00 | 0 | 0 | 0 | 0 | 0 | 0 | 0 | 0 |
| 0.33 | 6.20 | 0.33 | 1.13 | 0.33 | 0.44 | 0.33 | 0.12 | 0.33 | 0.10 |
| 0.67 | 9.03 | 0.67 | 1.55 | 0.67 | 0.64 | 0.67 | 0.19 | 0.67 | 0.17 |
| 1 | 11.29 | 1 | 1.87 | 1 | 0.76 | 1 | 0.29 | 1 | 0.24 |
| 1.5 | 14.27 | 1.5 | 2.31 | 1.5 | 0.99 | 1.5 | 0.39 | 1.5 | 0.33 |
| 2 | 16.39 | 2 | 2.86 | 2 | 1.25 | 2 | 0.52 | 2 | 0.42 |
| 3 | 20.25 | 3 | 3.67 | 3 | 1.65 | 3 | 0.71 | 3 | 0.56 |
| 4 | 23.40 | 4 | 4.39 | 4 | 2.04 | 4 | 0.91 | 4 | 0.73 |
| 5 | 25.94 | 5 | 5.09 | 5 | 2.40 | 5 | 1.10 | 5 | 0.88 |
| 6 | 28.14 | 6 | 5.74 | 6 | 2.76 | 6 | 1.32 | 6 | 1.02 |
| 7 | 30.16 | 7 | 6.37 | 7 | 3.12 | 7 | 1.50 | 7 | 1.18 |
| 9 | 33.51 | 9 | 7.51 | 9 | 3.80 | 9 | 1.87 | 9 | 1.48 |
| 11 | 36.47 | 11 | 8.65 | 11 | 4.48 | 11 | 2.25 | 11 | 1.79 |
| 13 | 39.05 | 13 | 9.67 | 13 | 5.08 | 13 | 2.63 | 13 | 2.07 |
| 16 | 41.92 | 16 | 11.23 | 16 | 5.97 | 16 | 3.22 | 16 | 2.48 |
| 19 | 44.58 | 19 | 12.64 | 19 | 6.81 | 19 | 3.79 | 19 | 2.93 |
| 22 | 46.76 | 22 | 14.07 | 22 | 7.72 | 22 | 4.36 | 22 | 3.41 |
| 26 | 49.31 | 27 | 16.21 | 27 | 8.90 | 27 | 5.17 | 27 | 4.06 |
| 32 | 51.76 | 33 | 18.28 | 33 | 10.14 | 33 | 6.06 | 33 | 4.82 |
| 39 | 54.01 | 40 | 20.78 | 40 | 11.68 | 40 | 7.21 | 40 | 5.71 |
| 47 | 56.38 | 46 | 22.89 | 46 | 13.09 | 46 | 8.26 | 46 | 6.58 |
| 54 | 58.13 | 53 | 24.86 | 53 | 14.55 | 53 | 9.28 | 53 | 7.49 |
| 61 | 59.69 | 60 | 27.00 | 60 | 16.00 | 60 | 10.42 | 60 | 8.37 |
| 68 | 61.12 | 67 | 28.75 | 67 | 17.20 | 67 | 11.37 | 67 | 9.13 |
| 75 | 62.36 | 74 | 30.54 | 74 | 18.49 | 74 | 12.20 | 74 | 10.03 |
| 82 | 63.57 | 81 | 32.38 | 81 | 19.79 | 81 | 13.11 | 81 | 10.83 |
| 89 | 64.56 | 89 | 34.18 | 89 | 21.11 | 89 | 14.15 | 89 | 11.68 |
| 95 | 65.41 | 96 | 35.79 | 96 | 22.26 | 96 | 14.99 | 96 | 12.48 |
| 102 | 66.32 | 103 | 37.20 | 103 | 23.35 | 103 | 15.80 | 103 | 13.21 |
| 109 | 67.19 | 110 | 38.68 | 110 | 24.40 | 110 | 16.72 | 110 | 13.99 |
| 116 | 67.88 | 117 | 40.23 | 117 | 25.53 | 117 | 17.55 | 117 | 14.90 |
| 123 | 68.69 | 123 | 41.61 | 123 | 26.63 | 123 | 18.43 | 123 | 15.67 |
| 129 | 69.38 | 130 | 43.07 | 130 | 27.70 | 130 | 19.31 | 130 | 16.49 |
| 136 | 70.02 | 137 | 44.66 | 137 | 28.90 | 137 | 20.29 | 137 | 17.42 |
| 143 | 70.66 | 144 | 46.38 | 144 | 30.25 | 144 | 21.36 | 144 | 18.53 |
| 150 | 71.15 | 151 | 47.94 | 151 | 31.67 | 151 | 22.48 | 151 | 19.53 |
| 157 | 71.65 | 158 | 49.69 | 158 | 33.09 | 158 | 23.67 | 158 | 20.71 |
| 164 | 72.08 | 166 | 51.55 | 166 | 34.44 | 166 | 24.99 | 166 | 21.88 |
| 172 | 72.55 | 174 | 53.09 | 174 | 35.79 | 174 | 26.15 | 174 | 23.02 |
| 179 | 72.99 | 182 | 54.79 | 182 | 37.22 | 182 | 27.34 | 182 | 24.35 |
| 186 | 73.36 | 190 | 56.37 | 190 | 38.60 | 190 | 28.66 | 190 | 25.62 |
| 193 | 73.72 | 198 | 57.54 | 198 | 39.86 | 198 | 29.80 | 198 | 26.70 |
| 200 | 74.09 | 206 | 58.78 | 206 | 41.32 | 206 | 31.00 | 206 | 27.87 |
| 206 | 74.40 | 214 | 60.03 | 214 | 42.66 | 214 | 32.08 | 214 | 29.08 |
| 213 | 74.70 | 222 | 61.18 | 222 | 43.91 | 222 | 33.25 | 222 | 30.22 |
| 220 | 75.04 | 230 | 62.12 | 230 | 44.94 | 230 | 34.12 | 230 | 31.19 |
| 227 | 75.37 | 238 | 62.97 | 238 | 46.07 | 238 | 35.11 | 238 | 32.19 |
| 234 | 75.68 | 246 | 63.74 | 246 | 47.07 | 246 | 35.98 | 246 | 33.04 |
| 241 | 76.00 | 254 | 64.61 | 254 | 48.33 | 254 | 37.02 | 254 | 34.02 |
| 249 | 76.36 | 262 | 65.54 | 262 | 49.11 | 262 | 37.97 | 262 | 34.89 |
| 257 | 76.63 | 270 | 66.56 | 270 | 49.86 | 270 | 38.95 | 270 | 35.79 |
| 265 | 76.90 | 278 | 67.54 | 278 | 50.60 | 278 | 39.99 | 278 | 36.79 |
| 273 | 77.12 | | | | | | | | |
| 281 | 77.30 | | | | | | | | |
| 289 | 77.50 | | | | | | | | |
| 297 | 77.70 | | | | | | | | |
| 305 | 77.84 | | | | | | | | |
| 313 | 77.99 | | | | | | | | |
| 321 | 78.08 | | | | | | | | |
| 329 | 78.21 | | | | | | | | |
| 337 | 78.29 | | | | | | | | |

2. Stent with Coating of Polyurethane from Example 3 (Comparative)

| | Basecoat (μg) | Sirolimus (μg) | Topcoat (μg) |
|---|---|---|---|
| Stent 1 | 1141 | 171.15 | 0 |
| Stent 2 | 1096 | 164.4 | 127 |
| Stent 3 | 1091 | 163.65 | 329 |
| Stent 4 | 1113 | 166.95 | 504 |
| Stent 5 | 1103 | 165.45 | 704 |

| | Stent 1 | | Stent 2 | | Stent 3 | | Stent 4 | | Stent 5 |
|---|---|---|---|---|---|---|---|---|---|
| | Total | | Total | | Total | | Total | | Total |
| Time | abs. | Time | abs | Time | abs. | Time | abs | Time | abs |
| (h) | (μg) | (h) | . (μg) | (h) | (μg) | (h) | (μg) | (h) | (μg) |
| 0.0 | 0 | 0 | 0 | 0 | 0 | 0 | 0 | 0 | 0 |
| 0.33 | 15.876 | 0.33 | 13.388 | 0.33 | 11.548 | 0.33 | 10.82 | 0.33 | 10.432 |
| 0.67 | 26.62 | 0.67 | 22.248 | 0.67 | 19.344 | 0.67 | 18.868 | 0.67 | 17.872 |
| 1 | 34.624 | 1 | 29.824 | 1 | 26.856 | 1 | 26.084 | 1 | 24.796 |
| 2 | 44.412 | 1.5 | 39.252 | 1.5 | 35.612 | 1.5 | 34.556 | 1.5 | 33.196 |
| 2 | 52.928 | 2 | 47.984 | 2 | 43.228 | 2 | 42.156 | 2 | 41.112 |
| 3 | 66.548 | 3 | 60.228 | 3 | 54.372 | 3 | 53.228 | 3 | 52.032 |
| 4 | 78.356 | 4 | 71.988 | 4 | 64.796 | 4 | 63.868 | 4 | 61.964 |
| 5 | 88.336 | 5 | 81.936 | 5 | 74.188 | 5 | 73.328 | 5 | 71.1 |
| 6 | 96.336 | 6 | 90.844 | 6 | 82.5 | 6 | 81.916 | 6 | 79.116 |
| 7 | 103.124 | 7 | 98.348 | 7 | 90.348 | 7 | 89.748 | 7 | 86.616 |
| 9 | 111.092 | 9 | 106.588 | 9 | 99.056 | 9 | 98.784 | 9 | 95.28 |
| 11 | 116.756 | 11 | 112.288 | 11 | 105.356 | 11 | 105.848 | 11 | 102.52 |
| 13 | 120.684 | 13 | 116.136 | 13 | 109.812 | 13 | 111.044 | 13 | 107.992 |
| 16 | 124.204 | 16 | 118.8 | 16 | 115.004 | 16 | 114.948 | 16 | 112.004 |
| 19 | 126.816 | 19 | 121.396 | 19 | 117.224 | 19 | 116.668 | 19 | 116.068 |
| 22 | 128.784 | 22 | 122.536 | 22 | 120.196 | 22 | 118.208 | 22 | 118.096 |
| 26 | 130.452 | 26 | 123.388 | 26 | 122.82 | 26 | 119.492 | 26 | 119.524 |
| 32 | 131.876 | 32 | 123.884 | 32 | 124.696 | 32 | 120.312 | 32 | 120.52 |
| 39 | 132.96 | 39 | 124.268 | 39 | 126.136 | 39 | 120.872 | 39 | 121.176 |
| 46 | 133.58 | 46 | 124.468 | 46 | 127.136 | 46 | 121.212 | 46 | 121.608 |
| 53 | 133.924 | 52 | 124.624 | 52 | 127.8 | 52 | 121.432 | 52 | 121.892 |
| 60 | 134.088 | 59 | 124.696 | 59 | 128.352 | 59 | 121.6 | 59 | 122.088 |
| 67 | 134.204 | 66 | 124.744 | 66 | 128.72 | 66 | 121.736 | 66 | 122.24 |
| 74 | 134.284 | 73 | 124.772 | 73 | 128.984 | 73 | 121.804 | 73 | 122.316 |
| 81 | 134.34 | 80 | 124.772 | 80 | 129.176 | 80 | 121.892 | 80 | 122.384 |
| | Stent 1 | | Stent 2 | | Stent 3 | | Stent 4 | | Stent 5 |
| Time | fraction | Time | fraction | Time | fraction | Time | fraction | Time | fraction |
| (h) | (wt. %) | (h) | (wt. %) | (h) | (wt. %) | (h) | (wt. %) | (h) | (wt. %) |
| 0 | 0 | 0 | 0 | 0 | 0 | 0 | 0 | 0 | 0 |
| 0.33 | 9.28 | 0.33 | 8.14 | 0.33 | 7.06 | 0.33 | 6.48 | 0.33 | 6.31 |
| 0.67 | 15.55 | 0.67 | 13.53 | 0.67 | 11.82 | 0.67 | 11.30 | 0.67 | 10.80 |
| 1 | 20.23 | 1 | 18.14 | 1 | 16.41 | 1 | 15.62 | 1 | 14.99 |
| 2 | 25.95 | 2 | 23.88 | 2 | 21.76 | 2 | 20.70 | 2 | 20.06 |
| 2 | 30.92 | 2 | 29.19 | 2 | 26.41 | 2 | 25.25 | 2 | 24.85 |
| 3 | 38.88 | 3 | 36.64 | 3 | 33.22 | 3 | 31.88 | 3 | 31.45 |
| 4 | 45.78 | 4 | 43.79 | 4 | 39.59 | 4 | 38.26 | 4 | 37.45 |
| 5 | 51.61 | 5 | 49.84 | 5 | 45.33 | 5 | 43.92 | 5 | 42.97 |
| 6 | 56.29 | 6 | 55.26 | 6 | 50.41 | 6 | 49.07 | 6 | 47.82 |
| 7 | 60.25 | 7 | 59.82 | 7 | 55.21 | 7 | 53.76 | 7 | 52.35 |
| 9 | 64.91 | 9 | 64.83 | 9 | 60.53 | 9 | 59.17 | 9 | 57.59 |
| 11 | 68.22 | 11 | 68.30 | 11 | 64.38 | 11 | 63.40 | 11 | 61.96 |
| 13 | 70.51 | 13 | 70.64 | 13 | 67.10 | 13 | 66.51 | 13 | 65.27 |
| 16 | 72.57 | 16 | 72.26 | 16 | 70.27 | 16 | 68.85 | 16 | 67.70 |
| 19 | 74.10 | 19 | 73.84 | 19 | 71.63 | 19 | 69.88 | 19 | 70.15 |
| 22 | 75.25 | 22 | 74.54 | 22 | 73.45 | 22 | 70.80 | 22 | 71.38 |
| 26 | 76.22 | 26 | 75.05 | 26 | 75.05 | 26 | 71.57 | 26 | 72.24 |
| 32 | 77.05 | 32 | 75.36 | 32 | 76.20 | 32 | 72.06 | 32 | 72.84 |
| 39 | 77.69 | 39 | 75.59 | 39 | 77.08 | 39 | 72.40 | 39 | 73.24 |
| 46 | 78.05 | 46 | 75.71 | 46 | 77.69 | 46 | 72.60 | 46 | 73.50 |
| 53 | 78.25 | 52 | 75.81 | 52 | 78.09 | 52 | 72.74 | 52 | 73.67 |
| 60 | 78.35 | 59 | 75.85 | 59 | 78.43 | 59 | 72.84 | 59 | 73.79 |
| 67 | 78.41 | 66 | 75.88 | 66 | 78.66 | 66 | 72.92 | 66 | 73.88 |
| 74 | 78.46 | 73 | 75.90 | 73 | 78.82 | 73 | 72.96 | 73 | 73.93 |
| 81 | 78.49 | 80 | 75.90 | 80 | 78.93 | 80 | 73.01 | 80 | 73.97 |

3. Stent with Coating of Polyurethane from Example 4 (Comparative)

| | Basecoat μg) | Sirolimus (μg) | Topcoat (μg) |
|---|---|---|---|
| Stent 1 | 1162 | 174.3 | 0 |
| Stent 2 | 1114 | 167.1 | 108 |
| Stent 3 | 1139 | 170.85 | 322 |
| Stent 4 | 1112 | 166.8 | 520 |
| Stent 5 | 1109 | 166.35 | 723 |

| | Stent 1 | | Stent 2 | | Stent 3 | | Stent 4 | | Stent 5 |
|---|---|---|---|---|---|---|---|---|---|
| | Total | | Total | | Total | | Total | | Total |
| Time | abs. | Time | abs. | Time | abs. | Time | abs. | Time | abs. |
| (h) | (μg) | (h) | (μg) | (h) | (μg) | (h) | (μg) | (h) | (μg) |
| 0 | 0 | 0 | 0 | 0 | 0 | 0 | 0 | 0 | 0 |
| 0.33 | 22.156 | 0.33 | 14.376 | 0.33 | 12.476 | 0.33 | 10.74 | 0.33 | 9.352 |
| 0.67 | 32.9 | 0.67 | 24.152 | 0.67 | 21.212 | 0.67 | 17.612 | 0.67 | 15.956 |
| 1 | 44.528 | 1 | 32.756 | 1 | 29.192 | 1 | 24.176 | 1 | 21.54 |
| 1.5 | 59.508 | 1.5 | 43.06 | 1.5 | 38.288 | 1.5 | 32.004 | 1.5 | 28.3 |
| 2 | 71.688 | 2 | 52.88 | 2 | 47.228 | 2 | 39.756 | 2 | 34.968 |
| 3 | 87.868 | 3 | 65.664 | 3 | 59.028 | 3 | 50.448 | 3 | 44.032 |
| 4 | 100.376 | 4 | 75.876 | 4 | 69.124 | 4 | 59.776 | 4 | 52.188 |
| 5 | 109.432 | 5 | 84.2 | 5 | 77.776 | 5 | 68.244 | 5 | 60.076 |
| 6 | 116.072 | 6 | 90.14 | 6 | 84.74 | 6 | 75.12 | 6 | 66.388 |
| 7 | 121.048 | 7 | 94.872 | 7 | 90.616 | 7 | 81.064 | 7 | 71.98 |
| 9 | 125.836 | 9 | 99.176 | 9 | 96.28 | 9 | 87.412 | 9 | 78.356 |
| 11 | 129.5 | 11 | 102.072 | 11 | 100.608 | 11 | 92.076 | 11 | 83.912 |
| 13 | 132.48 | 13 | 105.092 | 13 | 102.632 | 13 | 95.848 | 13 | 88.084 |
| 16 | 135.4 | 15 | 107.336 | 15 | 104.008 | 15 | 98.66 | 15 | 91.48 |
| 19 | 137.808 | 17 | 108.948 | 17 | 104.944 | 17 | 100.876 | 17 | 94.116 |
| 22 | 139.712 | 20 | 110.316 | 20 | 105.608 | 20 | 105.548 | 20 | 99.024 |
| 26 | 141.336 | 24 | 111.196 | 24 | 106.048 | 24 | 106.992 | 24 | 101.064 |
| 32 | 142.544 | 30 | 111.82 | 30 | 106.336 | 30 | 107.96 | 30 | 102.612 |
| 39 | 143.332 | 37 | 112.228 | 37 | 106.496 | 37 | 108.64 | 37 | 103.768 |
| 46 | 143.856 | 44 | 112.488 | 44 | 106.644 | 44 | 109.1 | 44 | 104.556 |
| 53 | 144.092 | 50 | 112.652 | 50 | 106.728 | 50 | 109.404 | 50 | 105.048 |
| 60 | 144.252 | 57 | 112.796 | 57 | 106.772 | 57 | 109.596 | 57 | 105.396 |
| 67 | 144.372 | 64 | 112.872 | 64 | 106.808 | 64 | 109.716 | 64 | 105.636 |
| 74 | 144.44 | 71 | 112.944 | 71 | 106.852 | 71 | 109.828 | 71 | 105.832 |
| 81 | 144.46 | 78 | 112.964 | 78 | 106.876 | 78 | 109.868 | 78 | 105.972 |
| | Stent 1 | | Stent 2 | | Stent 3 | | Stent 4 | | Stent 5 |
| Time | fraction | Time | fraction | Time | fraction | Time | fraction | Time | fraction |
| (h) | (wt. %) | (h) | (wt. %) | (h) | (wt. %) | (h) | (wt. %) | (h) | (wt. %) |
| 0 | 0 | 0 | 0 | 0 | 0 | 0 | 0 | 0 | 0 |
| 0.33 | 12.71 | 0.33 | 8.60 | 0.33 | 7.30 | 0.33 | 6.44 | 0.33 | 5.62 |
| 0.67 | 18.88 | 0.67 | 14.45 | 0.67 | 12.42 | 0.67 | 10.56 | 0.67 | 9.59 |
| 1 | 25.55 | 1 | 19.60 | 1 | 17.09 | 1 | 14.49 | 1 | 12.95 |
| 1.5 | 34.14 | 2 | 25.77 | 2 | 22.41 | 2 | 19.19 | 2 | 17.01 |
| 2 | 41.13 | 2 | 31.65 | 2 | 27.64 | 2 | 23.83 | 2 | 21.02 |
| 3 | 50.41 | 3 | 39.30 | 3 | 34.55 | 3 | 30.24 | 3 | 26.47 |
| 4 | 57.59 | 4 | 45.41 | 4 | 40.46 | 4 | 35.84 | 4 | 31.37 |
| 5 | 62.78 | 5 | 50.39 | 5 | 45.52 | 5 | 40.91 | 5 | 36.11 |
| 6 | 66.59 | 6 | 53.94 | 6 | 49.60 | 6 | 45.04 | 6 | 39.91 |
| 7 | 69.45 | 7 | 56.78 | 7 | 53.04 | 7 | 48.60 | 7 | 43.27 |
| 9 | 72.20 | 9 | 59.35 | 9 | 56.35 | 9 | 52.41 | 9 | 47.10 |
| 11 | 74.30 | 11 | 61.08 | 11 | 58.89 | 11 | 55.20 | 11 | 50.44 |
| 13 | 76.01 | 13 | 62.89 | 13 | 60.07 | 13 | 57.46 | 13 | 52.95 |
| 16 | 77.68 | 15 | 64.23 | 15 | 60.88 | 15 | 59.15 | 15 | 54.99 |
| 19 | 79.06 | 17 | 65.20 | 17 | 61.42 | 17 | 60.48 | 17 | 56.58 |
| 22 | 80.16 | 20 | 66.02 | 20 | 61.81 | 20 | 63.28 | 20 | 59.53 |
| 26 | 81.09 | 24 | 66.54 | 24 | 62.07 | 24 | 64.14 | 24 | 60.75 |
| 32 | 81.78 | 30 | 66.92 | 30 | 62.24 | 30 | 64.72 | 30 | 61.68 |
| 39 | 82.23 | 37 | 67.16 | 37 | 62.33 | 37 | 65.13 | 37 | 62.38 |
| 46 | 82.53 | 44 | 67.32 | 44 | 62.42 | 44 | 65.41 | 44 | 62.85 |
| 53 | 82.67 | 50 | 67.42 | 50 | 62.47 | 50 | 65.59 | 50 | 63.15 |
| 60 | 82.76 | 57 | 67.50 | 57 | 62.49 | 57 | 65.71 | 57 | 63.36 |
| 67 | 82.83 | 64 | 67.55 | 64 | 62.52 | 64 | 65.78 | 64 | 63.50 |
| 74 | 82.87 | 71 | 67.59 | 71 | 62.54 | 71 | 65.84 | 71 | 63.62 |
| 81 | 82.88 | 78 | 67.60 | 78 | 62.56 | 78 | 65.87 | 78 | 63.70 |

4. Stent with Coating of Polyurethane from Example 5 (Comparative)

| | Basecoat (μg) | Sirolimus (μg) | Topcoat (μg) |
|---|---|---|---|
| Stent 1 | 1100 | 165 | 0 |
| Stent 2 | 1076 | 161.4 | 105 |
| Stent 3 | 1107 | 166.05 | 317 |
| Stent 4 | 1125 | 168.75 | 528 |
| Stent 5 | 1092 | 163.8 | 731 |

| | Stent 1 | | Stent 2 | | Stent 3 | | Stent 4 | | Stent 5 |
|---|---|---|---|---|---|---|---|---|---|
| | Total | | Total | | Total | | Total | | Total |
| Time | abs. | Time | abs. | Time | abs. | Time | abs. | Time | abs. |
| (h) | (μg) | (h) | (μg) | (h) | (μg) | (h) | (μg) | (h) | (μg) |
| 0 | 0 | 0 | 0 | 0 | 0 | 0 | 0 | 0 | 0 |
| 0.33 | 12.06 | 0.33 | 12.508 | 0.33 | 10.452 | 0.33 | 11.168 | 0.33 | 9.544 |
| 0.67 | 17.456 | 0.67 | 21.228 | 0.67 | 13.528 | 0.67 | 17.648 | 0.67 | 15.964 |
| 1 | 21.448 | 1 | 29.432 | 1 | 20.116 | 1 | 23.676 | 1 | 21.84 |
| 1.5 | 26.98 | 1.5 | 39.232 | 1.5 | 27.664 | 1.5 | 30.64 | 1.5 | 27.992 |
| 2 | 31.616 | 2 | 47.26 | 2 | 34.688 | 2 | 37.316 | 2 | 34.032 |
| 3 | 39.736 | 3 | 58.892 | 3 | 44.792 | 3 | 47.708 | 3 | 42.816 |
| 4 | 46.876 | 4 | 69.048 | 4 | 53.908 | 4 | 56.572 | 4 | 51.084 |
| 5 | 52.672 | 5 | 77.824 | 5 | 62.288 | 5 | 64.796 | 5 | 58.692 |
| 6 | 58.244 | 6 | 85.344 | 6 | 69.76 | 6 | 72.06 | 6 | 65.316 |
| 7 | 63.268 | 7 | 91.856 | 7 | 76.5 | 7 | 78.912 | 7 | 71.288 |
| 9 | 71.652 | 9 | 99.976 | 9 | 85.712 | 9 | 87.468 | 9 | 78.92 |
| 11 | 78.852 | 11 | 106.62 | 11 | 93.788 | 11 | 94.804 | 11 | 86.088 |
| 13 | 84.968 | 13 | 111.544 | 13 | 99.752 | 13 | 100.548 | 13 | 93.152 |
| 16 | 92.912 | 16 | 115.092 | 16 | 102.992 | 15 | 105.304 | 15 | 97.936 |
| 19 | 100.072 | 19 | 117.628 | 19 | 105.904 | 17 | 109.708 | 17 | 101.964 |
| 22 | 105.332 | 22 | 119.46 | 22 | 107.368 | 20 | 111.716 | 20 | 104.704 |
| 26 | 110.788 | 26 | 121.06 | 26 | 108.404 | 24 | 115.696 | 24 | 108.412 |
| 32 | 115.772 | 32 | 122.24 | 32 | 109.052 | 30 | 119.196 | 30 | 111.408 |
| 39 | 120.584 | 39 | 122.692 | 39 | 109.916 | 37 | 122.14 | 37 | 113.74 |
| 46 | 123.676 | 46 | 123.32 | 46 | 110.172 | 44 | 124.128 | 44 | 115.28 |
| 53 | 126.168 | 52 | 123.712 | 52 | 110.344 | 50 | 125.564 | 50 | 116.284 |
| 60 | 127.828 | 59 | 124.072 | 59 | 110.424 | 57 | 126.728 | 57 | 117.112 |
| 67 | 129.076 | 66 | 124.352 | 66 | 110.54 | 64 | 127.68 | 64 | 117.804 |
| 74 | 129.884 | 73 | 124.544 | 73 | 110.58 | 71 | 128.424 | 71 | 118.24 |
| 81 | 130.556 | 80 | 124.732 | 80 | 110.624 | 78 | 128.96 | 78 | 118.596 |
| 88 | 130.988 | | | | | 84 | 129.392 | 84 | 118.88 |
| 95 | 131.368 | | | | | 91 | 129.648 | 91 | 119.064 |
| 102 | 131.692 | | | | | 98 | 129.916 | 98 | 119.256 |
| 109 | 131.932 | | | | | 105 | 130.036 | 105 | 119.396 |
| 116 | 132.12 | | | | | 112 | 130.2 | 112 | 119.492 |
| 123 | 132.264 | | | | | | | | |
| 130 | 132.348 | | | | | | | | |
| 137 | 132.46 | | | | | | | | |
| 144 | 132.54 | | | | | | | | |
| | Stent 1 | | Stent 2 | | Stent 3 | | Stent 4 | | Stent 5 |
| Time | fraction | Time | fraction | Time | fraction | Time | fraction | Time | fraction |
| (h) | (wt. %) | (h) | (wt. %) | (h) | (wt. %) | (h) | (wt. %) | (h) | (wt. %) |
| 0 | 0 | 0 | 0 | 0 | 0 | 0 | 0 | 0 | 0 |
| 0.33 | 7.31 | 0.33 | 7.75 | 0.33 | 6.29 | 0.33 | 6.62 | 0.33 | 5.83 |
| 0.67 | 10.58 | 0.67 | 13.15 | 0.67 | 8.15 | 0.67 | 10.46 | 0.67 | 9.75 |
| 1 | 13.00 | 1 | 18.24 | 1 | 12.11 | 1 | 14.03 | 1 | 13.33 |
| 1.5 | 16.35 | 1.5 | 24.31 | 1.5 | 16.66 | 1.5 | 18.16 | 1.5 | 17.09 |
| 2 | 19.16 | 2 | 29.28 | 2 | 20.89 | 2 | 22.11 | 2 | 20.78 |
| 3 | 24.08 | 3 | 36.49 | 3 | 26.98 | 3 | 28.27 | 3 | 26.14 |
| 4 | 28.41 | 4 | 42.78 | 4 | 32.46 | 4 | 33.52 | 4 | 31.19 |
| 5 | 31.92 | 5 | 48.22 | 5 | 37.51 | 5 | 38.40 | 5 | 35.83 |
| 6 | 35.30 | 6 | 52.88 | 6 | 42.01 | 6 | 42.70 | 6 | 39.88 |
| 7 | 38.34 | 7 | 56.91 | 7 | 46.07 | 7 | 46.76 | 7 | 43.52 |
| 9 | 43.43 | 9 | 61.94 | 9 | 51.62 | 9 | 51.83 | 9 | 48.18 |
| 11 | 47.79 | 11 | 66.06 | 11 | 56.48 | 11 | 56.18 | 11 | 52.56 |
| 13 | 51.50 | 13 | 69.11 | 13 | 60.07 | 13 | 59.58 | 13 | 56.87 |
| 16 | 56.31 | 16 | 71.31 | 16 | 62.02 | 15 | 62.40 | 15 | 59.79 |
| 19 | 60.65 | 19 | 72.88 | 19 | 63.78 | 17 | 65.01 | 17 | 62.25 |
| 22 | 63.84 | 22 | 74.01 | 22 | 64.66 | 20 | 66.20 | 20 | 63.92 |
| 26 | 67.14 | 26 | 75.01 | 26 | 65.28 | 24 | 68.56 | 24 | 66.19 |
| 32 | 70.16 | 32 | 75.74 | 32 | 65.67 | 30 | 70.63 | 30 | 68.01 |
| 39 | 73.08 | 39 | 76.02 | 39 | 66.19 | 37 | 72.38 | 37 | 69.44 |
| 46 | 74.96 | 46 | 76.41 | 46 | 66.35 | 44 | 73.56 | 44 | 70.38 |
| 53 | 76.47 | 52 | 76.65 | 52 | 66.45 | 50 | 74.41 | 50 | 70.99 |
| 60 | 77.47 | 59 | 76.87 | 59 | 66.50 | 57 | 75.10 | 57 | 71.50 |
| 67 | 78.23 | 66 | 77.05 | 66 | 66.57 | 64 | 75.66 | 64 | 71.92 |
| 74 | 78.72 | 73 | 77.16 | 73 | 66.59 | 71 | 76.10 | 71 | 72.19 |
| 81 | 79.12 | 80 | 77.28 | 80 | 66.62 | 78 | 76.42 | 78 | 72.40 |
| 88 | 79.39 | | | | | 84 | 76.68 | 84 | 72.58 |
| 95 | 79.62 | | | | | 91 | 76.83 | 91 | 72.69 |
| 102 | 79.81 | | | | | 98 | 76.99 | 98 | 72.81 |
| 109 | 79.96 | | | | | 105 | 77.06 | 105 | 72.89 |
| 116 | 80.07 | | | | | 112 | 77.16 | 112 | 72.95 |
| 123 | 80.16 | | | | | | | | |
| 130 | 80.21 | | | | | | | | |
| 137 | 80.28 | | | | | | | | |
| 144 | 80.33 | | | | | | | | |

5. Stent with Coating of Polyurethane from Example 6 (Comparative)

| | Basecoat (μg) | Sirolimus (μg) | Topcoat (μg) |
|---|---|---|---|
| Stent 1 | 1138 | 170.7 | 0 |

| | Stent 1 (no topcoat) |
|---|---|
| Time (h) | Total abs. (μg) |
| 0 | 0 |
| 0.33 | 26.32 |
| 0.67 | 42.396 |
| 1 | 54.628 |
| 1.5 | 69.044 |
| 2 | 80.432 |
| 3 | 95.484 |
| 4 | 106.52 |
| 5 | 114.588 |
| 6 | 120.588 |
| 7 | 125.24 |
| 9 | 133.22 |
| 11 | 137.94 |
| 13 | 141.832 |
| 16 | 145.364 |
| 19 | 148.132 |
| 22 | 150.256 |
| 25 | 151.82 |
| 29 | 153.208 |
| 35 | 154.424 |
| 42 | 155.156 |
| 49 | 155.712 |
| 56 | 156.044 |
| 63 | 156.252 |
| 70 | 156.452 |
| 78 | 156.584 |
| | Stent 1 (no topcoat) |
| Time (h) | fraction (wt. %) |
| 0 | 0.00 |
| 0.33 | 15.42 |
| 0.67 | 24.84 |
| 1 | 32.00 |
| 1.5 | 40.45 |
| 2 | 47.12 |
| 3 | 55.94 |
| 4 | 62.40 |
| 5 | 67.13 |
| 6 | 70.64 |
| 7 | 73.37 |
| 9 | 78.04 |
| 11 | 80.81 |
| 13 | 83.09 |
| 16 | 85.16 |
| 19 | 86.78 |
| 22 | 88.02 |
| 25 | 88.94 |
| 29 | 89.75 |
| 35 | 90.47 |
| 42 | 90.89 |
| 49 | 91.22 |
| 56 | 91.41 |
| 63 | 91.54 |
| 70 | 91.65 |
| 78 | 91.73 |

### 6. Discussion of Results

The objective of the development was to produce a stent coating which delivers active ingredient in continuous small doses over a number of weeks from the depot present in the coating.

The raw data can be interpreted as follows:

Example 2 (inventive): Release takes place over a long period. After 200 hours there is still a continuous release of sirolimus. The coating with an active ingredient-free polymer coat over the active ingredient-containing coat has a significant effect. By this means, the release rate is reduced further. After more than 200 hours, there is still continuous delivery of active ingredient, without the active ingredient depot having been used up.

Example 3 (comparative): There is rapid release. The active ingredient depot is used up after about 30 hours. The application of a drug-free topcoat produces no significant deceleration of release.

Example 4 (comparative): There is a rapid release. The active ingredient depot is used up after about 30 hours. The application of a drug-free topcoat does not substantially slow down the release. The topcoat prevents more than 70% of the active ingredient used being released.

Example 5 (comparative): Release is rapid. The active ingredient depot is used up after about 30 hours. The application of a drug-free topcoat has no significant slowing-down effect on release.

Example 6 (comparative): Release is very rapid. The amount of material released is substantially higher than with all the other stents. The active ingredient depot is exhausted after 20 hours.

The active ingredient depot is exhausted after not more than 30 hours for all comparative compounds.

1-17. (canceled)

18. A polyurethane urea comprising at least one structural unit of formula (I) 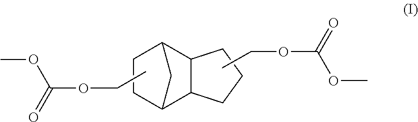
wherein said polyurethane urea is not terminated with at least one copolymer unit of polyethylene oxide and polypropylene oxide.

19. The polyurethane urea of claim 18, wherein said polyurethane urea is based on a polycarbonate polyol component which preferably has an average hydroxyl functionality of 1.7 to 2.3.

20. The polyurethane urea of claim 19, wherein said polycarbonate polyol component comprises a polycarbonate polyol a1) which are obtained by reaction of a carbonic acid derivative with a difunctional alcohol of formula (II)

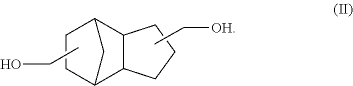

21. The polyurethane urea of claim 20, wherein said polycarbonate polyol component further comprises a polycarbonate polyols a2).

22. The polyurethane urea of claim 21, wherein said polycarbonate polyol a2) comprises a compound which has an average hydroxyl functionality of from 1.7 to 2.3 and a molecular weight, as determined by the OH number, of from 400 to 6000 g/mol and is based on hexane-1,6-diol, butane-1,4-diol, or mixtures thereof.

23. The polyurethane urea of claim 18, wherein said polyurethane urea has a number-average molecular weight of from 1,000 to 100,000 g/mol as measured in dimethylacetamide at 30° C.

24. The polyurethane urea of claim 18, wherein said polyurethane urea comprises active pharmacological ingredients.

25. A substrate having applied thereon a basecoat comprising the polyurethane urea of claim 18.

26. The substrate of claim 25, wherein from the basecoat a topcoat is applied which comprises the polyurethane urea of claim 18, and which differs in its chemical and/or physical properties from the basecoat.

27. The substrate of claim 26, wherein the basecoat comprises an active pharmacological ingredient.

28. The substrate of claim 26, wherein the topcoat is free of active ingredient.

29. The substrate of claim 26, wherein the basecoat has a coat thickness of from 5 to 20 μm and/or the topcoat has a coat thickness of from 0.5 to 10 μm.

30. The substrate of claim 26, wherein the substrate is a medical article.

31. A layer structure comprising at least one active ingredient-containing layer comprising a polyurethane urea of claim 24 and at least one active ingredient-free layer comprising a polyurethane urea comprising at least one structural unit of formula (I) 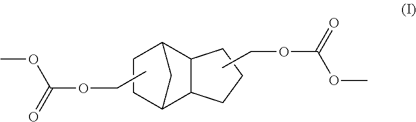
wherein said polyurethane urea is not terminated with at least one copolymer unit of polyethylene oxide and polypropylene oxide.

32. A method for coating a substrate, comprising applying at least one layer comprising the polyurethane urea of claim 18 to the substrate.

33. The method of claim 32, wherein a basecoat comprising an active ingredient-containing polyurethane urea as claimed in claim 7 is applied to the substrate, and a topcoat comprising an active ingredient-free polyurethane urea comprising at least one structural unit of formula (I) 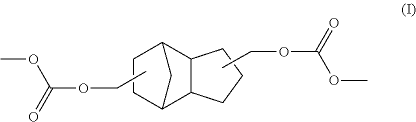
wherein said polyurethane urea is not terminated with at least one copolymer unit of polyethylene oxide and polypropylene oxide
is applied to the basecoat.

34. A coated substrate obtained by the method of claim 32.

35. The substrate of claim 26, wherein the substrate is an implantable article.

36. The substrate of claim 26, wherein the substrate is a stent.
